(19) United States
(12) Patent Application Publication
(10) Pub. No.: US 2026/0207896 A1
(43) Pub. Date: Jul. 23, 2026

(54) GUIDING SHEATH, SHEATH SYSTEMS, AND METHODS OF USE

(71) Applicant: Route 92 Medical, Inc., San Mateo, CA (US)

(72) Inventors: Andrew Messenger, San Mateo, CA (US); John Miller, San Mateo, CA (US)

(21) Appl. No.: 19/451,407

(22) Filed: Jan. 16, 2026

Related U.S. Application Data
(60) Provisional application No. 63/746,747, filed on Jan. 17, 2025.

(51) Int. Cl. A61M 25/06; A61B 17/22; A61L 29/04; A61M 5/00; A61M 25/00; A61M 25/01; A61M 39/06

(52) U.S. Cl. CPC A61M 25/0662; A61B 17/22; A61L 29/04; A61M 5/007; A61M 25/0045; A61M 25/005; A61M 25/0097; A61B 2017/22079; A61M 2025/0098; A61M 25/0108; A61M 2025/0681; A61M 39/06; A61M 2039/062

(57) ABSTRACT

An intravascular guiding sheath for facilitation of intraluminal medical procedures within the neurovasculature including a variable stiffness sheath body having a single working lumen and a length between a proximal opening and a distal opening. The sheath body has a wall with an outer jacket layer, an inner liner layer, and a reinforcement layer between the outer jacket layer and the inner liner layer. The reinforcement layer includes a coil reinforcement wound over the inner liner layer and a braid reinforcement positioned directly onto the coil reinforcement that has a variable pic count. The outer jacket layer has a plurality of segments along the length of the sheath body, each segment of the plurality of segments having a durometer and arranged so that moving proximally along the length of the sheath body the durometer of the plurality of segments increases. Related devices, systems, and methods are provided.

### CROSS-REFERENCE TO RELATED APPLICATIONS

This application claims the benefit of priority under 35 U.S.C. § 119 (e) to U.S. Provisional Patent Application Ser. No. 63/746,747, filed Jan. 17, 2025. The disclosure of the patent application is incorporated by reference herein in its entirety.

### FIELD

The present technology relates generally to a guiding sheath, sheath systems, and methods of use.

### BACKGROUND

Acute ischemic stroke (AIS) usually occurs when an artery to the brain is occluded, preventing delivery of fresh oxygenated blood from the heart and lungs to the brain. These occlusions are typically caused by a thrombus or an embolus lodging in the artery and blocking the artery that feeds a territory of brain tissue. If an artery is blocked, ischemia follows, and brain cells may stop working. Furthermore, if the artery remains blocked for more than a few minutes, the brain cells may die, leading to permanent neurological deficit or death. Therefore, immediate treatment is critical.

The endovascular treatments for AIS face the challenge of navigating complex anatomy leading to the vessels in the brain. The anatomy leading to the lesion can be tortuous and diseased, which may complicate device delivery. To access the cerebral anatomy, guide catheters or guiding sheaths are used to direct interventional devices, such as retrievable structures, guidewires, microcatheters, and intermediate access catheters to the target site from an access site. It can often be very challenging to access and establish guiding sheath position in a fashion that is stable and provides support for device delivery. To maneuver the catheters into position, coaxial, triaxial, or quadraxial systems are often used in which a guidewire/microcatheter system is first deployed and coaxial larger catheters are subsequently delivered. The clinical challenge, especially in the octogenarian population, is the elongation of the aortic arch against the fixed thoracic descending aorta, leading to a shifting of all great vessels, especially the brachiocephalic takeoff. Such shifting makes it more challenging to access the anatomy during treatment of, e.g., stroke, aneurysm, and other distally-located vascular diseases. As catheters, wires, balloons, stents, or retrievable structures are advanced through the great vessels, they have a tendency to prolapse into the ascending aorta when pushed into a highly angulated and/or tortuous anatomy.

There is a need for guiding sheaths and sheath systems that have improved deliverability to access and provide support for endovascular treatment systems at distal sites in the neurovasculature.

### SUMMARY

In an implementation, provided is an intravascular guiding sheath for facilitation of intraluminal medical procedures within a neurovasculature. The guiding sheath includes a variable stiffness sheath body having a single working lumen extending through the sheath body from a proximal end region to a distal opening at a distal end region of the sheath body, the sheath body having a length between a proximal opening and the distal opening, wherein the working lumen has a minimum inner diameter that is at least 0.080″; and a proximal hub luer at the proximal end region of the sheath body. The sheath body has a wall having a wall thickness from the proximal end region to the distal end region. The wall has an outer jacket layer, an inner liner layer, and a reinforcement layer between the outer jacket layer and the inner liner layer. The reinforcement layer has a coil reinforcement wound over the inner liner layer and a braid reinforcement positioned directly onto the coil reinforcement that has a variable pic count. The outer jacket layer has a plurality of outer jacket segments along the length of the sheath body, each outer jacket segment of the plurality of outer jacket segments having a durometer and arranged so that moving proximally along the length of the sheath body the durometer of the plurality of outer jacket segments increases.

The plurality of outer jacket segments can include at least six outer jacket segments along the length of the sheath body. The at least six outer jacket segments can include a distal set of outer jacket segments and a proximal set of outer jacket segments. The distal set of outer jacket segments can include four outer jacket segments, each of the four outer jacket segments has a length. The length of each of the four outer jacket segments can be substantially the same length to each other. The proximal set of outer jacket segments can include at least two outer jacket segments, each of the at least two outer jacket segments has a length. The length of each of the at least two outer jacket segments can be longer than the length of each of the four outer jacket segments.

The inner liner layer and the outer jacket layer can be polymeric and the reinforcement layer is metal. The coil reinforcement can be stainless steel. The braid reinforcement can include at least four zones of different density including a first braid zone along the distal end region of the sheath body, a second braid zone located proximal to the first braid zone, a third braid zone located proximal to the second braid zone, and a fourth braid zone located proximal to the third braid zone and extending along the proximal end region of the sheath body. Each of the first braid zone, second braid zone, third braid zone, and fourth braid zone has a pics per inch (PPI). The PPI can increase by about 30% between the fourth braid zone and the third braid zone. The PPI can increase by about 10% between the third braid zone and the second braid zone. The PPI can increase by about 10% between the second braid zone and the first braid zone.

The wall thickness can be substantially uniform. The wall thickness can be about 0.006″ to about 0.012″. The guiding sheath can further include a double layer strain relief along a junction between the proximal end region of the sheath body and the proximal hub luer. The double layer strain relief can include an outer layer formed of a first polymer material and an inner layer formed of a second polymer material.

In an interrelated implementation, provided is an intravascular guiding sheath for facilitation of intraluminal medical procedures within a neurovasculature. The guiding sheath includes a variable stiffness sheath body having a single working lumen extending through the sheath body from a proximal end region to a distal opening at a distal end region of the sheath body. The sheath body has a length between a proximal opening and the distal opening and the working lumen has a minimum inner diameter that is at least 0.080″. The guiding sheath includes a proximal hub luer at the proximal end region of the sheath body for connection to a fluid delivery or aspiration source. The sheath body includes a wall having a wall thickness. The wall has an outer jacket layer, an inner liner layer, and a reinforcement layer between the outer jacket layer and the inner liner layer. The reinforcement layer includes a stainless-steel coil wound over the inner liner layer and a braid positioned directly onto the coil that has a variable pic count. The outer jacket layer has a plurality of outer jacket segments along the length of the sheath body, each outer jacket segment of the plurality of outer jacket segments having a durometer and arranged so that moving proximally along the length of the sheath body the durometer of the plurality of outer jacket segments increases. The braid includes at least three braid zones having different pics per inch (PPI) from one another. A distal-most braid zone of the at least three braid zones is overlaid by at least three outer jacket segments of the plurality of outer jacket segments of the outer jacket layer.

The plurality of outer jacket segments can be at least six outer jacket segments along the length of the sheath body. The at least six outer jacket segments can include a distal set of outer jacket segments and a proximal set of outer jacket segments. The distal set of outer jacket segments can include four outer jacket segments, each of the four outer jacket segments having a length, wherein the length of each of the four outer jacket segments can be substantially the same length to each other. The proximal set of outer jacket segments can include at least two outer jacket segments, each of the at least two outer jacket segments has a length. The length of each of the at least two outer jacket segments can be longer than the length of each of the four outer jacket segments. The wall thickness can be substantially uniform. The wall thickness can be about 0.006″ to about 0.012″. The guiding sheath can further include a double layer strain relief along a junction between the proximal end region of the sheath body and the proximal hub luer. The double layer strain relief can include an outer layer formed of a first polymer material and an inner layer formed of a second polymer material.

In an interrelated implementation, provided is an intravascular guiding sheath for facilitation of intraluminal medical procedures within a neurovasculature that includes a variable stiffness sheath body having a single working lumen extending through the sheath body from a proximal end region to a distal opening at a distal end region of the sheath body. The sheath body has a length between a proximal opening and the distal opening, wherein the working lumen has a minimum inner diameter. A proximal hub luer is at the proximal end region of the sheath body. The sheath body includes a wall having a wall thickness from the proximal end region to the distal end region. The wall includes an outer jacket layer, an inner liner layer, and a reinforcement layer between the outer jacket layer and the inner liner layer. The reinforcement layer includes a coil reinforcement wound over the inner liner layer and a braid reinforcement positioned directly onto the coil reinforcement that has a variable pic count. The outer jacket layer has at least six outer jacket segments along the length of the sheath body, each outer jacket segment of the at least six outer jacket segments having a durometer and arranged so that moving proximally along the length of the sheath body the durometer of the outer jacket segments increases. The variable stiffness sheath body can be 6 French up to 10 French. The braid reinforcement can compress the coil reinforcement against the inner liner layer. A distal portion of the sheath body can have a kink resistance as measured by vise jaw gap distance that is no greater than 40 mm, no greater than 35 mm, or no greater than 30 mm.

Any of the intravascular guiding sheaths described herein can be part of a system incorporating any of the navigation catheters described herein. Any of the intravascular guiding sheaths described herein can be part of a system incorporating one or more of a navigation catheter, an aspiration catheter, or other catheter.

The details of one or more variations of the subject matter described herein are set forth in the accompanying drawings and the description below. Other features and advantages of the subject matter described herein will be apparent from the description and drawings and from the claims. While certain features of the currently disclosed subject matter are described for illustrative purposes, it should be readily understood that such features are not intended to be limiting. The claims that follow the disclosure are intended to define the scope of the protected subject matter.

### BRIEF DESCRIPTION OF THE DRAWINGS

These and other aspects will now be described in detail with reference to the following drawings. Generally, the figures are not to scale in absolute terms or comparatively, but are intended to be illustrative. Also, relative placement of features and elements may be modified for the purpose of illustrative clarity.

FIG. 1A is a side view of an implementation of a guiding sheath;

FIG. 1B is a detailed, cross-sectional view of a distal tip of the guiding sheath of FIG. 1A;

FIG. 2 is an implementation of the guiding sheath of FIG. 1A in a sheath system of optional components for delivery of the guiding sheath to a target location;

FIGS. 3A-3B illustrate braid reinforcement layers for the guiding sheath;

FIGS. 4A-4B illustrate the dual-layer strain relief for the guiding sheath;

FIG. 5 shows bending stiffness profile of the guiding sheath of FIG. 1A having a braid over a coil and a greater number of material transitions along its length compared to a conventional guiding sheath having a coil over a braid and fewer material transitions along its length.

It should be appreciated that the drawings are for example only and are not meant to be to scale. The drawings are intended to be illustrative to dimensions and are not to scale in absolute terms or comparatively. It is to be understood that devices described herein may include features not necessarily depicted in each figure.

### DETAILED DESCRIPTION

Neurovascular guiding sheaths, which are sometimes referred to as guide sheaths guide catheters, or access catheters are used to facilitate passage of other large-bore catheters, aspiration catheters, stent retrievers, coils, flow diverters, and other neurovascular devices from an access site of a patient and direct them up into vessels leading to the brain. Guiding sheaths are preferably soft-tipped to avoid damaging fragile vasculature, trackable and pushable to navigate tortuous anatomy, and designed to support delivery of other longer devices through the guiding sheath into distal sites without kinking or collapse. Guiding sheaths are introduced into access sites that are remote from the treatment site, such as the femoral, radial, ulnar, or brachial arteries and veins. The length of the guiding sheath is determined by the distance between the access site and the desired location of the distal tip suitable to support a catheter intended for delivery to distal treatment sites. The working lumen of the guiding sheaths are preferably large enough to accept large-bore devices and catheter systems for delivery to the distal treatment sites. Many interventional devices and catheters for mechanical thrombectomy in distal intracranial vessels are referred to as “superbore” or “super large-bore” catheters have larger inner diameters (e.g., 0.088″ or above) with correspondingly larger outer diameters. The guiding sheaths to accept these larger-sized catheters and catheter systems are suitably sized.

Introducers for inserting a guiding sheath into a vessel are relatively short catheters (e.g., about 10 cm-25 cm long) that are sized based on the outer diameter of a catheter that will fit through the sheath or the inner diameter of the introducer. Introducers for inserting guiding sheaths are generally less than 10 Fr (e.g., about 3.3 mm OD) and preferably no greater than about 9 Fr (e.g., about 3.0 mm ID) so that the introducer is large enough to receive the outer diameter of the guiding sheath, but not so large that its outer diameter increases the risk of complications at the vessel access site. The outer diameter of introducers can vary, but generally a 9 Fr introducer has an outer diameter less than 4.0 mm (e.g., no greater than about 3.8 mm). Larger introducers (e.g., 10 Fr or 3.4 mm ID having an outer diameter of about 4.0 mm or greater) can pose risks of bleeding, hematoma, pseudoaneurysm, arteriovenous fistula, and vessel trauma and other complications. Maximizing the inner diameter of the guiding sheath while minimizing the outer diameter so that the guiding sheath can be advanced through smaller introducers and thereby reduce the access site penetration is beneficial to supporting the advancement of large-sized catheters to distal sites while reducing patient risk of complications.

Described herein are guiding sheaths designed to introduce therapeutic or diagnostic devices, catheters, and catheter systems into the vasculature from an arterial or venous access site that provide the trackability and flexibility characteristics to navigate through tortuous anatomy while maintaining the kink-resistance and proximal support to advance a variety of catheters and catheter systems through the guiding sheath and minimizing wall thickness of the wall of the sheath body to avoid patient complications at the access site. The guiding sheaths described herein preferably have an outer diameter (OD) sized to insert through a 9 Fr introducer and have an inner diameter (ID) sized to receive superbore catheters. In turn, the guiding sheaths described herein have thin catheter body walls (e.g., less than about 0.50 mm (0.020″)) and preferably less than about 0.45 mm (0.018″), and more preferably less than 0.42 mm (0.017″) that are reinforced so as to prevent kinking and proximal prolapse during use.

The guiding sheaths described herein are preferably useful for introduction of interventional devices into the neurovasculature. Where implementations are described herein with specific regard to accessing a neurovascular anatomy, the systems, catheters, devices, and methods described herein should not be limited to this and may also be applicable to other uses. For example, the catheter systems described herein may be used to deliver working devices to an extracranial vessel including the carotid vessels leading to the cerebral anatomy, or a target vessel of a coronary anatomy, peripheral anatomy, or other vasculature anatomy. Coronary vessels are considered herein including left and right coronary arteries, posterior descending artery, right marginal artery, left anterior descending artery, left circumflex artery, M1 and M2 left marginal arteries, and D1 and D2 diagonal branches. Any of a variety of peripheral vessels are considered herein including the popliteal arteries, anterior tibial arteries, dorsalis pedis artery, posterior tibial arteries, and fibular artery. Any of a variety of venous targets are considered herein including intracranial veins and venous sinuses.

Where the phrase “guiding sheath” or “guide sheath” or “guide” or “sheath” or “access sheath” is used herein, such a device may be used for insertion of other catheters into the distal vasculature through the lumen of the sheath. “Sheaths” are conventionally sized according to their inner diameter whereas “catheters” are conventionally sized according to their outer diameter. The term “catheter” may be used herein to refer to any tubular device having an inner diameter and an outer diameter. “Catheter” may be used interchangeably herein with the term “sheath”. Neither “catheter” nor “sheath” is used herein in a manner that is intended to be limiting to any specific use. For example, where a catheter is described as being used for access, other purposes besides or in addition to access is considered herein, such as the delivery of fluids to a treatment site or for the removal of fluids from a treatment site, such as by aspiration through the sheath or catheter. Alternatively, the catheters described herein may also be useful for access to other parts of the body outside the vasculature.

As used herein, “embolus” or “embolus material” or “embolic material” or “embolic region” refers to material within a zone of an occlusion site that is denser or a relatively hard consistency that is preferably placed in contact with a distal end of an aspiration catheter to successfully perform aspiration embolectomy. The embolus may be a thrombus (a clot of blood) or other material that formed at a first blood vessel location (e.g., a coronary vessel), breaks loose, and travels through the circulation to a second blood vessel location. As used herein, “in situ thrombus” or “thrombus material” or “thrombotic material” or “thrombotic region” or “in situ clot material” or “clot material” refers to material within a zone of an occlusion site that accumulates in situ at the site of the embolus and is often less dense or relatively soft and fluid-like. As used herein, “organized thrombus” refers to in situ thrombus material or clot material that accumulates at the site of embolus and is denser and less fluid-like than the in situ clot material.

As used herein, “an occlusion” or “an occlusion site” or “occlusive material” refers to the blockage that occurred as a result of an atherosclerotic lesion or embolus lodging within a vessel and disrupting blood flow through the vessel or a stenosis within a vessel or sinus. The occlusion or occlusive material can include both thrombus and embolus as well as another non-thrombotic narrowing of the vessel.

As used herein, “an aneurysm” refers to the ballooning out of a weakened section of vessel wall. A “cerebral aneurysm” or “intracranial aneurysm” refers to an aneurysm in a vessel of the brain.

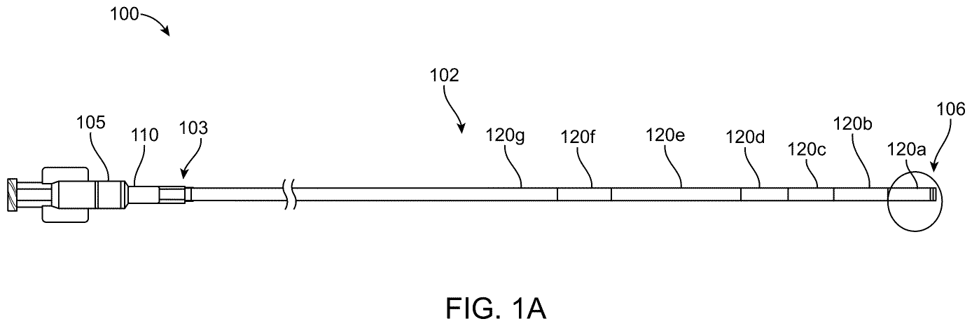

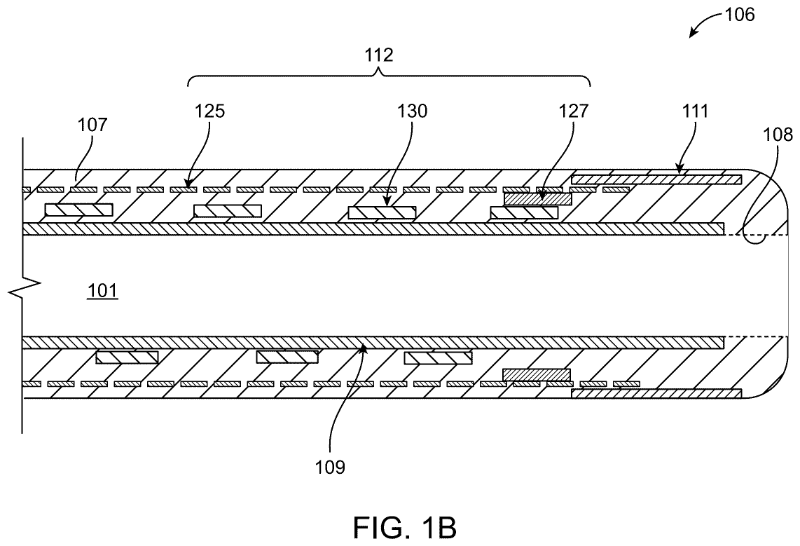

Turning now to the figures, an implementation of an intravascular access guiding sheath 100 for facilitation of intraluminal medical procedures within the neurovasculature is illustrated in FIGS. 1A-1B. The guiding sheath 100 is a long sheath with a variable stiffness sheath body 102. A single, working lumen 101 extends through the sheath body 102 from a proximal end region 103 to a distal opening 108 at a distal end region of the sheath body 102. The working lumen 101 is sized to insert and advance another catheter or catheters. The working lumen 101 is configured to receive any of a variety of catheters therethrough such that a distal end of the catheter can extend beyond a distal end of the guiding sheath 100 through the distal opening 108. The working lumen 101 is preferably the only lumen extending through the tubular, sheath body 102 although a multi-lumen sheath body 102 is considered as well. For example, the sheath body 102 can additionally incorporate a separate guidewire lumen that is smaller and separated from the working lumen 101. At least the distal end region of the sheath body 102 is designed with trackability and flexibility characteristics to assume and navigate the bends of tortuous vasculature without kinking, collapsing, or causing vascular trauma, even, for example, when subjected to high aspiration forces. The sheath body 102 is designed along the proximal end region with strength and stiffness characteristics to be pushable and supportive of a variety of large-bore catheters and catheter systems while minimizing wall thickness to avoid patient complications at the access site. As will be described in more detail below, the dual-layer strain relief and tight-pitch reinforcement provides the access sheath with both distal and proximal support during advancement, for example, to the carotid bulb, even with a substantially thinner wall. A plurality of different copolymer zones create a smooth overall transition profile from a softer distal tip 106 to a stiff proximal end region for seamless deliverability and stability.

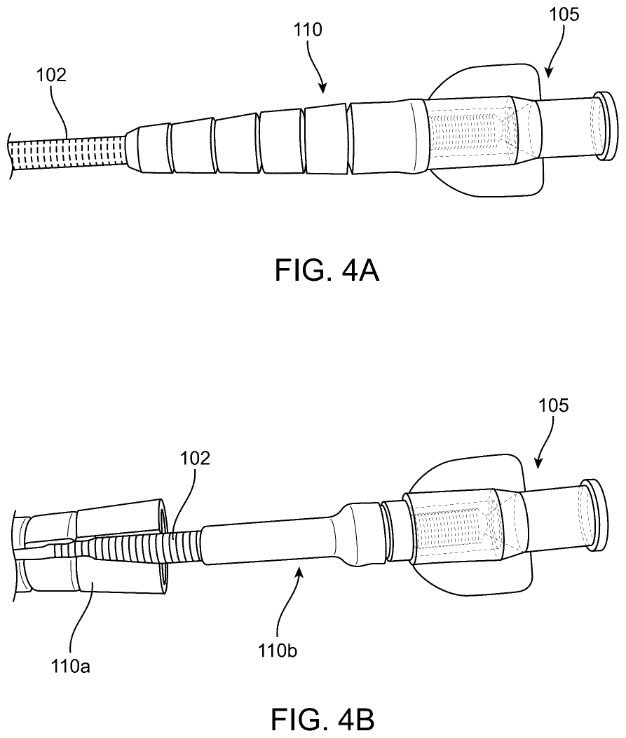

Still with respect to FIGS. 1A-1B, the sheath body 102 can incorporate a proximal hub luer 105 at a proximal end region 103 for connection of fluid delivery and/or fluid removal systems (see also FIGS. 4A-4B). The hub luer 105 can be sized to allow a catheter or other device pass through it for insertion of the device into the working lumen 101 of the sheath body 102. For example, the hub luer 105 can include a through-hole that is about 0.105″ to about 0.110″, preferably at least 0.106″ to about 0.108″. Superbore catheters having lumens of 0.088″ and greater typically have an outer diameter that are about 2.6 mm-2.74 mm. The hub luer 105 inner diameter (and the inner diameter of the working lumen 101 of the sheath body) is preferably sized to receive the outer diameter of these superbore catheters.

FIGS. 4A-4B illustrate how the proximal hub luer 105 is reinforced by a double layer strain relief. A double layer, strain relief element 110 at the junction between the tubular, sheath body 102 and the hub luer 105 is affixed to the proximal end of the sheath body 102. The strain relief element 110 can be attached over the junction between the hub luer 105 and sheath body 102 and aids in preventing the collapse and/or kinking of the working lumen 101 at or near this junction. The strain relief element 110 spreads bending force along a greater length and away from the specific connection between the hub luer 105 and the proximal end of the sheath body 102. An outer layer 110a of the strain relief element 110 is formed of a first polymer material or materials and an inner layer 110b of the strain relief element 110 is formed of a second polymer material or materials. The first polymer material of the outer layer 110a can be generally soft and flex fatigue resistant material, such as a thermoplastic vulcanizate (TPV). The second polymer material of the inner layer 110b can be flexible and able to conform to irregular shape yet provide greater strain relief, such as a polyolefin shrink tubing. The first polymer material(s) of the outer layer 110a can be softer, harder, or the same softness compared to the second polymer material(s) of the inner layer 110b. The inner layer 110b can be heated and shrunk down and the outer layer 110a placed over the shrunk inner layer 110b. The inner layer 110b can have a thickness of about 3/16″ (0.188″ or 4.78 mm) with a minimum expanded inner diameter of about 0.188″ (4.78 mm) and a maximum recovered inner diameter of about 0.093″ (2.36 mm). The strain relief element 110 can have an external shape and outer dimension that provides an outer surface that is ergonomical and provides heft suitable for catheter manipulations. The external shape of the strain relief element 110 can be cylindrical, conical, spheroid, or egg-shaped. The outer diameter of the strain relief element 110 moving distally tapers to approach 0.122″ to 0.126″ (3.10 mm-3.20 mm) or the outer diameter of the proximal end of the tubular, sheath body 102. The outer diameter of the strain relief element moving proximally approaches 0.286″ to 0.292″ (7.26 mm-7.42 mm) or the maximum outer diameter of the hub luer 105.

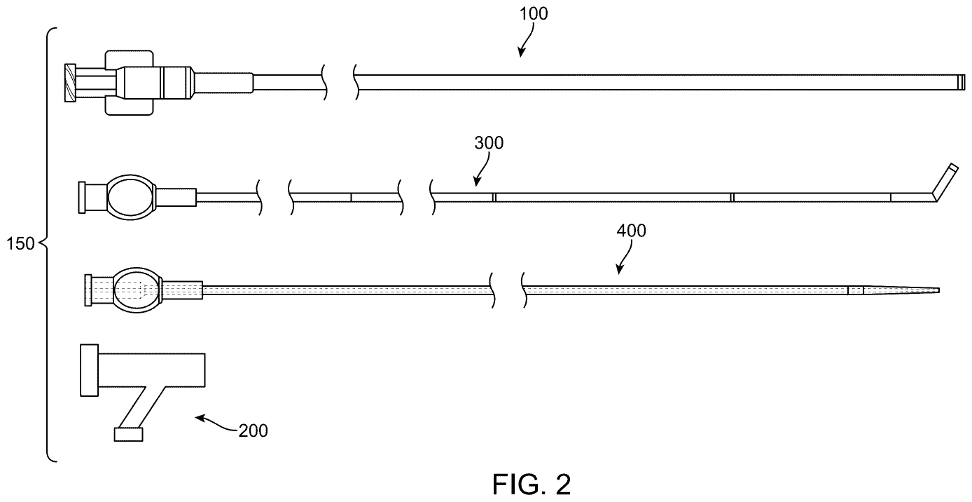

The hub luer 105 can be integral with or designed to couple to a hemostasis valve component 200 (see FIG. 2), such as a passive seal valve, a Tuohy Borst valve or a rotating hemostatic valve (RHV), including a dual RHV or a multi-head RHV. The proximal hemostasis valve component 200 may include one or more lumens molded into a connector body to connect to the working lumen 101 of the sheath body 102 of the guiding sheath 100. The hemostasis valve component 200 can be constructed of thick-walled polymer tubing or reinforced polymer tubing. The hemostasis valve component 200 allows for the introduction of devices through the guiding sheath 100 into the vasculature, while preventing or minimizing blood loss and preventing air introduction into the guiding sheath 100. The hemostasis valve component 200 can have an adjustable opening that is open large enough to allow removal of devices that have adherent clot without causing the clot to dislodge at the hemostasis valve component 200 during removal. Alternately, the hemostasis valve component 200 can be removable, such as when a device is being removed from the guiding sheath 100, to prevent clot dislodgement at the hemostasis valve component 200.

The hemostasis valve component 200 can form a Y-connector on the proximal end region 103 of the guiding sheath 100 such that the first port of the hemostasis valve component 200 can be used for insertion of a catheter into the working lumen 101 of the guiding sheath 100 and a second port of the hemostasis valve component 200 can be used for another purpose. For example, a syringe or other device can be connected at the second port via a connector to deliver a forward drip, a flush line for contrast or saline injections through the sheath body 102 toward the distal tip 106 and into the target anatomy. The hemostasis valve component 200 can also connect to a large-bore aspiration line and an aspiration source (not shown) such as a syringe or pump to draw suction through the working lumen. The aspiration source can be an active source of aspiration such as an aspiration pump, a regular or locking syringe, a hand-held aspirator, hospital suction, or the like, configured to draw suction through the working lumen. The aspiration source can be a locking syringe (for example a VacLok syringe) attached to a flow controller. The hemostasis valve component 200 can also allow the guiding sheath 100 to be flushed with saline or radiopaque contrast during a procedure.

Contrast agent can be injected through the guiding sheath 100 into the vessel to visualize the occlusion site by angiogram. For example, the guiding sheath 100 can be positioned so that at least a portion is positioned within the carotid artery. The contrast agent may be injected through the guiding sheath 100 once positioned in this location. Contrast agent can also be injected through one or more catheters inserted through the guiding sheath 100. A baseline angiogram can be obtained, for example in the anterior/posterior (AP) and/or lateral views, prior to device insertion to assess occlusion location by injection of contrast media through the guiding sheath 100 with fluoroscopic visualization. Fluoroscopic visualization may continue as the catheter system is advanced and subsequent angiograms can be captured periodically to assess reperfusion. The baseline angiogram image can be superimposed, such as with digital subtraction angiography, so that the vasculature and/or treatment site are visible while the catheter system is advanced.

Again with respect to FIG. 1A, the length of the sheath body 102 is configured to allow the distal tip 106 of the sheath body 102 to be positioned at a location sufficient to support catheters and devices being advanced to distal sites while the proximal end region extends outside the vessel access site. The length between the proximal end and the distal-most end of the sheath body 102 can allow for advancing from the access site (e.g., femoral artery) to the internal carotid artery (ICA) including the petrous portion of the ICA, for example, with additional length providing for adjustments if needed. In some implementations, the length of the sheath body 102 can be in the range of about 70 cm to about 100 cm and preferably about 80 cm to about 90 cm and an overall length including the proximal hub luer 105 that is about 75 cm to about 110 cm and preferably about 85 cm to about 100 cm. In one example, the length of the sheath body, also called the working length, is provided in multiple lengths for selection by a user of 80 cm and 90 cm with corresponding full lengths of 86 cm and 96 cm. The sheath body 102 can be longer, for example, about 80 cm up to about 100 cm or up to about 105 cm or up to about 120 cm total. The point of insertion for the guiding sheath 100 can vary including femoral, carotid, radial, brachial, ulnar, or subclavian arteries and veins, as well as direct puncture of the carotid artery or jugular vein. The lengths of the sheath body 102 described herein can be modified to accommodate different access points for the guiding sheath 100. For example, a sheath body 102 of a guiding sheath 100 for entry through the femoral artery near the groin may be longer than a sheath body 102 of a guiding sheath 100 for entry through the subclavian artery.

Now with respect to FIG. 1B, the sheath body 102 of the guiding sheath 100 can be radiopaque in one or more regions so that it is visible under fluoroscopy. For example, the guiding sheath 100 can include one or more radiopaque markers 111. At least one radiopaque marker 111 can be disposed near the distal opening 108. The distance between the distal-most terminus of the sheath body 102 to the distal radiopaque marker 111 can be no greater than about 0.8 mm, preferably about 0.3 mm to about 0.8 mm. The radiopaque marker 111 can be swaged, painted, embedded, or otherwise disposed in or on the sheath body 102. In some implementations, the radiopaque marker(s) 111 includes a barium polymer, tungsten polymer blend, tungsten-filled or platinum-filled marker that maintains flexibility of the devices and improves transition along the length of the component and its resistance to kinking. In some implementations, the radiopaque marker(s) 111 is a tungsten-loaded PEBAX (a polyether-based polyamide) or polyurethane that is heat welded to the component. The radiopaque marker 111 can be a band of radiopaque material. In some implementations, the radiopaque marker(s) 111 is a 90% platinum/10% iridium marker band that is fully encapsulated between a liner and an outer jacket layer of the sheath body 102. In some implementations, the radiopaque marker(s) 111 include platinum, gold, tantalum, tungsten or any other substance visible under an x-ray fluoroscope.

The radiopaque markers 111 are shown in the figures as rings around a circumference of one or more regions of the sheath body 102. For example, the radiopaque marker 111 can be rolled to an inner diameter for positioning on the tubular, sheath body 102 (e.g., about 0.113″). However, the radiopaque markers 111 can have other shapes or create a variety of patterns that provide orientation to an operator regarding the position of the distal opening 108 within the vessel. Accordingly, an operator may visualize a location of the distal opening 108 under fluoroscopy to confirm that the distal opening 108 is directed toward a target anatomy where a catheter is to be delivered. For example, radiopaque marker(s) 111 allow an operator to rotate the sheath body 102 of the guiding sheath 100 at an anatomical access point, e.g., a groin of a patient, such that the distal opening provides access to an ICA by subsequent working device(s), e.g., catheters and wires advanced to the ICA. Any of the various components of the systems described herein can incorporate radiopaque markers.

The guiding sheath 100 is at least 6 French up to 10 French or larger to accept various-sized working devices, preferably, the guiding sheath has a 7 French, 8 French, or 9 French inner diameter. French scale for a guiding sheath is relevant to its inner diameter. For example, a 6 French guiding sheath 100 working lumen 101 has an inner diameter sized to accommodate 6 French catheters (1.98 mm or 0.078″ OD). The guiding sheath 100 can accommodate at least 6.3 French catheters (2.079 mm or 0.082″ OD), at least 7 French catheters (2.31 mm or 0.091″ OD), at least 8 French catheters (2.64 mm or 0.104″ OD), or larger catheters. The working lumen 101 preferably has a minimum inner diameter that is at least about 0.080″ up to about 0.115″, preferably about 0.090″ up to about 0.111″, and more preferably about 0.104″ up to about 0.108″ near the distal end. Such a working lumen 101 is suitable for use with any of a variety of catheters including aspiration catheters and support catheters having an outer diameter of less than or equal to about 0.105″ and a working length greater than or equal to about 100 cm. Superbore catheters having lumens of 0.088″ and greater typically have an outer diameter that are about 2.6 mm-2.74 mm. The inner diameter of the working lumen 101 of the sheath body 102 is preferably sized to receive the outer diameter of these superbore catheters.

The working lumen 101 of the guiding sheath 100 can extend from a proximal opening at the hub luer 105 to the distal opening 108 of the distal tip 106. The working lumen 101 can be designed to be generally uniform in inner diameter along its entire length between the proximal opening to the distal opening 108. The working lumen 101 can be designed to step-down in inner diameter along its length from the proximal opening to the distal opening 108 such that the inner diameter nearer to the proximal opening is larger than the inner diameter nearer to the distal opening 108. Regardless of the length and inner diameter, the guiding sheath 100 is resistant to kinking during distal advancement through the vasculature.

Introducers for access vessels can vary, but generally a 9 Fr introducer having an outer diameter that is less than 4.0 mm (e.g., no greater than about 3.8 mm) is preferred. The outer diameter of the sheath body 102 is preferably sized to insert through a 9 Fr introducer. The outer diameter of the sheath body 102 can be designed to be generally uniform along its entire length from a location near the proximal hub luer 105 to the distal tip 106. The presence of the radiopaque marker(s) 111 may locally increase the outer diameter of the sheath body 102 compared to the outer diameter on either side of the radiopaque marker 111 even where the outer diameter along the entire length of the sheath body 102 is designed to be substantially uniform. In some implementations, the outer diameter of the sheath body 102 can be designed to taper gradually towards the distal tip 106 or can be designed to step-down at a location between the proximal end and the distal tip 106 such that the distal outer diameter is smaller than the proximal outer diameter. Whether the outer diameter is substantially uniform along its length of changes along its length, the inner diameter along the distal end region of the sheath body 102 can be at least about 0.106″ to about 0.125″ and the outer diameter along the distal end region can be about 0.120″ to about 0.125″.

The distal tip 106 of the guiding sheath 100 can have the same or similar outer diameter as a section of the sheath body 102 leading up to the distal tip 106. Accordingly, the distal tip 106 may have a distal face orthogonal to a longitudinal axis passing through the sheath body 102 and the distal face may have an outer diameter substantially equal to a cross-sectional outer dimension of the sheath body 102. In an implementation, the distal tip 106 includes a chamfer, fillet, or taper, making the distal face diameter slightly less than the cross-sectional dimension of the sheath body 102. In a further implementation, the distal tip 106 may be an elongated tubular portion extending distal to a region of the sheath body 102 having a uniform outer diameter such that the elongated tubular portion has a reduced diameter compared to the uniform outer diameter of the sheath body 102. Thus, the distal tip 106 can be elongated or can be more bluntly shaped. Accordingly, the distal tip 106 may be configured to smoothly track through a vasculature and/or to dilate vascular restrictions as it tracks through the vasculature. The working lumen 101 may have a distal end forming the distal opening 108.

As described elsewhere herein, constraints such as minimum inner diameter sized to receive superbore catheters and maximum outer diameter to minimize access site punctures result in the guiding sheaths described herein being generally thin walled. The wall thickness of the sheath body 102 wall can be about 0.005″ to about 0.020″, preferably about 0.006″ to about 0.012″, most preferably about 0.008″. The wall thickness of the sheath body 102 between the proximal end and the distal end is preferably less than about 0.012″, less than about 0.0115″, less than about 0.011″, less than about 0.0105″, less than about 0.010″, less than about 0.0095″, less than about 0.009″, less than about 0.0085″ down to about 0.008″ while the inner diameter of the sheath body 102 between the proximal end and the distal end is at least about 0.095″ and preferably at least 0.106″. The proximal end outer diameter can be reduced (e.g., about 0.122″) by reducing the wall thickness (e.g., about 0.008″) while the inner diameter remains substantially large (e.g., about 0.106″) for receiving larger interventional devices. The thinner wall and lower profile allows for a smaller insertion hole through the vessel without impacting overall lumen size. In some implementations, the wall thickness of the guiding sheath 100 can slowly step down to be thinner towards a distal end of the sheath body 102 compared to a proximal end.

Despite the thin wall of sheath body 102 providing the overall lower profile, the guiding sheath 100 has improved deliverability, kink-resistance, kink diameter, torque, proximal column strength, and distal flexibility. The distal end region of the sheath body 102 is flexible for navigating tortuous anatomy (e.g., brachiocephalic take-off from the aortic arch into the common carotid artery) and gradually transitions to a stiffer proximal end region that provides better support and push response. The flexibility characteristics of the guiding sheath 100 are measurable using a 3-point Bending Test method (see Example 1). The guiding sheath 100 has a lower average delivery and withdrawal force compared to other guiding sheaths as measured via Track Testing method (see Example 2) indicating better trackability. The guiding sheath 100 has a higher kink resistance as measured by a vise jaw gap test (see Example 3) indicating an ability to form into tighter curves without kinking and/or collapse of the lumen and overall better navigability to distal sites in the neurovasculature.

The stiffness profile of the guiding sheath 100 can be over 5× more flexible within the distal-most 2.5 cm compared to more proximal regions (e.g., greater than about 25-26 cm away from the distal-most end). In an implementation of the sheath having 5 transitions in jacket materials within the distal-most 10-15 cm, the stiffness of the sheath is about 4× times the stiffness of the distal end. In another implementation of the sheath, the stiffness at the proximal end is about 27% greater than the stiffness of the sheath at the distal-most 10-15 cm. The total difference between the distal flexibility and the proximal flexibility can be greater than 5×.

For a guiding sheath 100 having a working length of 85-95 cm, the distal-most 2.5 cm of the guiding sheath 100 can be over 5× more flexible than the proximal-most segment of the guiding sheath 100, over 4× more flexible than the proximal-most 70 cm, over 3.5× more flexible than the proximal-most 82 cm, and at least 3× more flexible than the proximal-most 84 cm. Similarly, the average flexibility of the distal-most 10 cm of the guiding sheath 100 can be about 2.5× greater than the average flexibility of the proximal-most 70 cm of the guiding sheath 100. For a guiding sheath 100 having a working length of 85-95 cm, the proximal-most segment of the guiding sheath 100 having a material Shore hardness greater than 72D can be over 5× stiffer than the distal-most 2.5 cm of the guiding sheath 100 having a material Shore hardness no greater than about 25D. The proximal segment of the guiding sheath having a material Shore hardness that is about 72D can be over 4× stiffer than the distal-most 2.5 cm of the guiding sheath 100 having a material Shore hardness no greater than about 25D. The proximal segment of the guiding sheath having a material Shore hardness that is about 63D can be over 3.5× stiffer than the distal-most 2.5 cm of the guiding sheath 100 having a material Shore hardness no greater than about 25D. The proximal segments of the guiding sheath having a material Shore hardness that is about 55D can be at least 3× stiffer than the distal-most 2.5 cm of the guiding sheath 100 having a material Shore hardness no greater than about 25D. Similarly, the average flexibility of the proximal three segments having a material Shore hardness greater than about 55D can be about 2.5× less than the average flexibility of the distal-most 10 cm of the guiding sheath 100 having a material Shore hardness no greater than about 55D.

The guiding sheath 100 can be delivered to an intracranial vessel while maintaining an average maximum delivery force in grams that is less than about 800, less than about 750, less than about 700, or less than about 650, down to about 600. The average maximum withdrawal force in grams of the guiding sheath 100 can be kept to less than about 700, less than about 650, or less than about 600, down to about 590.

The kink resistance of the guiding sheath 100 within the distal end region (e.g., distal-most 10-15 cm) having the braid reinforcement positioned over the coil reinforcement (i.e., coil-under-braid arrangement) is greater than the kink resistance of a guiding sheath with the coil reinforcement over the braid reinforcement (i.e., coil-over-braid arrangement). The kink resistance can be about 10% to about 75% greater in the coil-under-braid arrangement guiding sheath compared to the coil-over-braid arrangement guiding sheath. The kink resistance can be at least 10%, at least 20%, at least 30%, at least 40%, at least 50%, at least 60%, up to about 75% greater in the coil-under-braid arrangement guiding sheath compared to the coil-over-braid arrangement guiding sheath. The kink resistance as measured by vise jaw gap distance can be no greater than about 45 mm, no greater than about 40 mm, and preferably no greater than about 30 mm. The coil-under-braid arrangement guiding sheaths can achieve a tighter curved shape without kinking compared to coil-over-braid arrangement guiding sheaths.

Again with respect to FIG. 1B, the improved deliverability, kink-resistance, kink diameter, torque, proximal support, and distal flexibility of the guiding sheath 100 described herein is due in part to the design of the reinforcement and polymer layout. The guiding sheath 100 has a multi-layered, variable stiffness sheath body 102 that includes an outer jacket layer 107, a lubricious, inner liner layer 109, and a reinforcement layer 112 sandwiched between the outer jacket layer 107 and the inner liner layer 109. The reinforcement layer 112 incorporates two reinforcement structures that are arranged relative to one another and relative to the sheath body 102 wall in a manner that provides specific advantages given the constraints of the thin wall resulting from large inner diameter and small outer diameter. Each will be described in more detail below.

The inner liner layer 109 can be constructed from a low friction polymer such as PTFE (polytetrafluoroethylene) or FEP (fluorinated ethylene propylene) to provide a smooth surface for the advancement of devices through the inner lumen. In a preferred implementation, the inner liner layer 109 is etched PTFE having a wall thickness of about 0.0005″ to about 0.0015″, or about 0.0010″. The inner liner layer 109 can run an entire length of the working lumen 101 of the sheath body 102 from the proximal opening to the distal opening 108. Alternatively, the inner liner layer 109 can terminate a short distance, such as about 0.5 mm-2.0 mm, from the distal opening 108.

The outer jacket layer 107 can provide mechanical integrity to the inner liner layer 109 and can be constructed from one or more materials, such as PEBAX, thermoplastic polyurethane, polyethylene, nylon, or the like. The outer jacket layer 107 of the sheath body 102 can be formed of increasingly softer materials towards the distal end. For example, a proximal region of the sheath body 102 can incorporate outer jacket layer segments formed of a material such as Nylon. Another region of the sheath body 102 can incorporate outer jacket layer segments having a hardness of 72D whereas areas more distal regions of the sheath body 102 can incorporate outer jacket layer segments that are increasingly more flexible and formed of materials having a hardness of 55D, 45D, 35D moving distally. The distal tip 106 can include an outer jacket layer segment(s) formed of a material having a hardness of no more than 35D and in some implementations softer than 35D.

The outer jacket layer 107 preferably incorporates a plurality of outer jacket segments 120 that are formed of materials selected to provide proximal stiffness and distal flexibility. Too few transitions in outer jacket segments creates step-changes in flexibility moving distally along the catheter than can increase the risk of kinking. Too many transitions in outer jacket segments can create a proximal section that is too stiff and/or brittle and a distal section that may also fail to keep structural integrity. Increasing the number of outer jacket segments can be challenging from a manufacturing standpoint and introduces potential kink points and/or defects, and may cause distal end regions that have impaired structural integrity of the lumen. The sheath body 102 is preferably formed to have between about 6 to 8 outer jacket segments 120 to transition from the stiffer proximal end region to the more flexible distal end region. FIG. 1A illustrates an implementation of a guiding sheath 100 having a plurality of outer jacket segments 120. The first segment 120a along the distal-most region of the sheath body 102 can be formed of 25D Pebax, the second segment 120b proximal to the first segment 120a can be 35D Pebax, the third segment 120c proximal to the second segment 120b can be 45D Pebax, the fourth segment 120d proximal to the third segment 120c can be 55D Pebax, the fifth segment 120e proximal to the fourth segment 120d can be 63D Pebax, the sixth segment 120f proximal to the fifth segment 120e can be 72D Pebax, and the seventh segment 120g proximal to the sixth segment 120f can be Nylon.

The guiding sheath 100 in the implementation of FIG. 1A has seven segments 120 of different materials along its length. The plurality of outer jacket segments 120 can include at least six segments or at least seven segments, etc. along the length of the sheath body. The specific lengths of each segment 120 can vary depending on the total working length of the guiding sheath 100 as can the specific number of outer jacket segments 120 from the distal end to the proximal end of the sheath body 102. The outer jacket segments can include a distal set of outer jacket segments and a proximal set of outer jacket segments. As an example, the sheath body 102 can include at least six outer jacket segments and the distal set of outer jacket segments including four outer jacket segments and the proximal set of segments includes at least two outer jacket segments. Each of the outer jacket segments has a length. The length of each of the four outer jacket segments in the distal set of outer jacket segments can be substantially the same length as one another. The length of each of the at least two outer jacket segments in the proximal set of outer jacket segments can be longer than the length of each of the four outer jacket segments in the distal set of outer jacket segments.

Again with respect to FIG. 1A described above, the first, second, third, and fourth outer jacket segments 120a-120d can be formed of a material having a Shore hardness equal to or less than about 55D Shore hardness and can each be about 20-30 mm long. The fifth and sixth outer jacket segments 120e-120f can be formed of a material having a Shore hardness greater than 55D Shore hardness, for example about 63D Shore hardness to about 72D Shore hardness, and can extend along a greater length than the more distal outer jacket segments, such as about 35 mm-125 mm. In an implementation, the fifth outer jacket segment 120e has a length of about 115-125 mm and the sixth outer jacket segment 120f has a length that is about 35-45 mm. The seventh outer jacket segment 120g can be even longer and form a majority of the stiffer proximal end region of the sheath body 102. The seventh outer jacket segment 120g can have a length of about 655-665 mm, depending on the total working length of the sheath body 102. The total working length of the sheath body 102 can be customized by changing a length of the last outer jacket segment (e.g., the seventh outer jacket segment). The total working length of the sheath body 102 in the implementation described above with respect to FIG. 1A is at least about 70 cm up to about 100 cm. The total working length of the sheath body 102 is suitable for insertion from a femoral artery access site. For a sheath body 102 intended for insertion from a radial artery access site, the total working length may be 70 to 100 cm. The sheath body 102 may still include at least 6 or at least seven outer jacket segments, but each outer jacket segment length as described above may be reduced by about 10%.

The number of transitions along the length of the sheath body 102 is preferably at least 5 outer jacket segments 120, at least 6 outer jacket segments 120, or at least 7 outer jacket segments 120. Increasing the number of outer jacket segments 120 and thus, the transition zones along the length of the sheath body 102 provides a more gradual transition profile between the soft, atraumatic distal tip 106 and the stiff, pushable proximal end for better navigation and delivery. The extruded outer jacket segments 120 can be cut and laminated to the sheath body 102. The outer jacket segments 120 can be laminated at an angle (e.g., about 30-degree angle). One or more of the outer jacket segments 120 along the length of the sheath body 102 can incorporate a radiopaque material, such as 20% Barium Sulfate as well as materials providing UV and heat stabilization.

As discussed elsewhere herein, the guiding catheters 100 are relatively thin-walled to overcome the constraints of having a large inner diameter to advance superbore catheters and a small outer diameter to minimize access site penetrations. The sheath body 102 can be circumferentially reinforced with a reinforcement layer 112 that preferably incorporates two reinforcement structures that are arranged relative to one another and relative to the sheath body 102 wall in a manner that provide better kink resistance, kink diameter, and deliverability given the constraints of the thin wall. The reinforcement layer 112 between the inner liner layer 109 and the outer jacket layer 107 prevents flattening and kinking of the working lumen 101 of the sheath body 102 during use thereby allowing unimpeded device navigation through bends in the vasculature as well as for applying aspiration or reverse flow forces without collapse. The reinforcement layer 112 can be made from one or more metals such as stainless steel, Nitinol, Nitinol braid, helical ribbon, helical wire, cut stainless steel, or the like, or stiff polymer such as PEEK. The reinforcement layer 112 can include a structure such as a coil and/or a braid, or stiff tubing that has been laser-cut or machine-cut so as to be flexible. In a preferred implementation, the reinforcement layer 112 is a braid overlaying a coil.

In another implementation, the reinforcement layer 112 is a hypotube such as a Nitinol (nickel titanium alloy) hypotube, stainless steel, or rigid polymer, or the like that is cut along at least a portion of its length to impart flexibility properties. The hypotube can have an inner diameter of about 0.095″ to about 0.125″. The hypotube can incorporate cuts along at least a portion of its length, preferably within the distal end region. In some implementations, a proximal end region of the hypotube is a hypotube without interruptions through its sidewall for stiffness and the distal end region incorporates interruptions (e.g., slots, cuts, perforations, etc.) for flexibility. Preferably, both the proximal end region and the distal end region of the hypotube incorporates cuts through the sidewall in one or more locations. The cuts can be designed to provide a transition from the stiffness of the proximal end region of the hypotube to the flexibility of the distal portion of the catheter or can transition to a distal portion of the catheter reinforced with another type of reinforcement (e.g., coil).

The cuts in the hypotube to impart flexibility can be perpendicular, substantially perpendicular, or angled with respect to the longitudinal axis of the tube. In a preferred implementation, the cuts extend in a non-perpendicular direction relative to the longitudinal axis of the hypotube. The cuts can create continuous spiral patterns, interrupted spiral patterns, interlocking spirals, hinged patterns and the like. The cuts can be straight cuts, slots, angled cuts, and intricate patterns. A pattern of the cuts preferably change over the length of the tube. In an implementation, the pattern of the cuts can change continuously over a length of the tube or over a portion of the length of the tube toward its distal end. For example, the cuts can be closer together for greater flexibility the further distal along the hypotube such that regions more proximally have cuts that are further apart than regions more distally so that the distal end region has a greater flexibility. The cuts can overlap to a greater degree near the distal end of the tube.

In an implementation, the cuts in the distal end region of the hypotube have different patterns in each of a plurality of tube segments. The different patterns formed by the cuts can have a pitch between about 0.001″ and about 0.05″, preferably about 0.003″ and about 0.016″. The segments can be constant in pitch along the length of the distal end region of the hypotube. The segments preferably vary in pitch along the length of the distal end region of the hypotube. The segments can have 2, 3, 4, or more different pitches along the length of a cut section of hypotube. The pitch can increase over the length towards the distal-most end of the hypotube.

In one example, a first section of the plurality of segments starting near a distal end of the hypotube can have a first pitch, which varies along the first section between about 0.003″-0.0045″, a second section of the plurality of segments moving proximally can have a longitudinally varying second pitch of between about 0.0045″-0.008″, a third section of the plurality of segments moving proximally can have a third pitch varying between about 0.008″-0.015″, and a fourth section of the plurality of segments moving proximally can have a varying or substantially constant fourth pitch of about 0.015″.

FIG. 1A illustrates an implementation of the sheath body 102 having a reinforcement layer 112 that includes a combination of a braid reinforcement 125 and a coil reinforcement 130. The braid reinforcement 125 is preferably positioned directly onto the coil reinforcement 130 (i.e., towards the external surface of the catheter), the coil reinforcement 130 being wound over the inner liner layer 109. In some implementations, an additional layer, such as a polymer layer or coating, can be positioned over the coil reinforcement 130 so as to lie between the inner coil reinforcement 130 and the braid reinforcement 125. Braids are more likely to disrupt coils during catheter constructions. Thus, where braids and coils are used together in a catheter it is conventional for braids to be laid down first over the inner liner layer 109 and the coil over the braid. Positioning the braid closer to the inner diameter can result in the braid becoming partially ovalize under the coil when the catheter bends, such as when navigating tight curves. The braid reinforcement 125 of the reinforcement layer 112 described herein, in contrast, enhances the torque characteristics of the sheath body 102, supports the column strength, and resists buckling or kinking even when navigating tight curves. The coil reinforcement 130 of the reinforcement layer 112 also can provide good kink resistance and flexibility. Positioning the coil reinforcement 130 under the braid reinforcement 125 means the coil reinforcement 130 lies closer to the inner diameter (i.e., centerline) of the sheath body 102 allowing for the most articulation before kinking. The braid reinforcement 125 positioned over the coil reinforcement 130 adds support to the articulation and further prevents kinking. The braid reinforcement 125 “cinches down” and compresses the coil reinforcement 130 against the inner liner layer 109 providing an overall smaller outer diameter to the sheath body wall. In some implementations, the wire selected for the braid reinforcement 125 can be thicker compared to a “braid-first” or “coil-over-braid” arrangement of reinforcement layer where no “cinching” of the coil reinforcement 130 is achieved. Thicker wires for the braid reinforcement 125 can provide better kink resistance at smaller kink diameters. A coil reinforcement over the braid reinforcement would have no “cinching” to reduce the overall outer diameter of the sheath body wall. Other techniques to reduce the outer diameter of the sheath body wall would be relied upon, such as using a thinner wire for the braid reinforcement 125. But thinner braid wires may not provide the same mechanical advantages provided by a thicker braid wire in terms of kink resistance. Positioning the coil closer to the inner diameter, as in the present guiding sheaths, prevents any deformation of the braid and the catheter. The braid outside the coil constrains the coil better, which provides better coil/braid structural integrity and performance, due to the potential to increase braid density (ppi) and winding force.

The braid reinforcement 125 is preferably stainless-steel 0.001″ round wire, such as 304V stainless steel. A round wire having a thickness that is less than 0.003″, less than 0.002″ down to about 0.001″ minimizes the overall outer diameter of the sheath body wall suitable for insertion through a 9 Fr introducer while the inner diameter remains sized sufficient to receive superbore catheters and provides sufficient cinching force against the coil reinforcement 130 under the braid wires. The wire for the braid reinforcement 125 is preferably round to provide better kink resistance, although the wire can be flat having a rectangular or square cross-sectional shape (e.g., 0.007″×0.003″ wire). The coil reinforcement 130 is preferably stainless-steel wire, such as 304V stainless steel ribbon having a width and a thickness. The coil ribbon can have a thickness that is greater than the braid wire thickness or diameter. In some implementations, the coil ribbon has a width that is about 4-8 times the thickness of the braid wire. For example, where the coil ribbon is 0.012″×0.002″ stainless steel ribbon, the coil has a width of 0.012″ and a thickness of 0.002″. The width of the coil ribbon can be about 12 times a thickness of the braid wire that is 0.001″ thick. The thickness of the coil ribbon can be about 2 times the thickness of the braid wire. The thickness of the coil ribbon and the braid wire together is less than about 0.005″ and preferably less than about 0.004″. Other materials for the coil reinforcement 130 are considered as well, such as Nitinol (e.g., Nitinol #1 SE ribbon coil). The coil reinforcement 130 can be wound right-hand or left-hand to have a pitch that is about 0.023″-0.027″, preferably about 0.025″.

The density of the braid reinforcement 125 and the thickness of the braid wires may be varied to adjust the bending, buckling, and torsional stiffness of the sheath body 102 at various sections, but is generally between 50 and 120 pics per inch (PPI) depending on the location along the length of the sheath body 102. The braid reinforcement 125 can be formed by 16 strands of wire having a pattern of 2 wires over and 2 wires under. Other implementations can include the pattern of 1 wire under 2 wires and 1 wire over 2 wires. The braid reinforcement 125 preferably extends from the hub luer 105 at the proximal end of the working lumen 101 to the location of the distal radiopaque marker 111. Thus, the braid reinforcement 125 can extend along a length of the sheath body 102 that is about 80-90 cm less the final few millimeters distal to the radiopaque marker 111.

The flexibility of the sheath body 102 can vary over its length, with increasing flexibility towards the distal portion of the sheath body 102. The variability in flexibility may be achieved in various ways. For example, the outer jacket layer 107 can change in durometer and/or material at various segments 120, as described above, with lower durometer outer jacket materials used in a distal section of the sheath body 102 compared to more proximal sections of the sheath body 102. The wall thickness of the outer jacket material may be reduced moving distally. The outer jacket material of the more distal segments can be lower durometer and/or thinner compared to the outer jacket material of the more proximal segments which can be higher durometer and/or thicker. The density of the reinforcement layer 112 may be varied to increase the flexibility in more distal regions of the sheath body 102. For example, the pitch of the coil reinforcement 130 may be stretched out in more distal regions compared to more proximal regions. The braid reinforcement 125 may have a higher pic count in more distal regions compared to more proximal regions. Combinations of the jacket segment material, jacket segment thickness, coil pitch, and/or braid pic count can be selected to provide a desired distal flexibility and proximal stiffness. In implementations where the reinforcement layer 112 comprises a cut tube, the cut pattern in the tubing may be varied to be more flexible in more distal regions compared to proximal regions. Alternately, the reinforcement layer 112 or the materials may change over the length of the sheath body 102. Any of a combination of these structural differences can be combined to achieve different flexibility along the distal portion of the sheath body 102 compared to the proximal portion of the sheath body 102. As an example, the sheath body 102 can including a coil reinforcement 130 wound over the inner liner layer 109, a braid reinforcement 125 is tensioned directly over the coil reinforcement 130, and a plurality of outer jacket layer segments 120 (e.g., at least six segments) along a length of the sheath body 102 between a proximal opening into the working lumen 101 to the distal opening 108 from the working lumen 101. Each segment 120 of the outer jacket layer 107 can be formed of a polymer material or materials that has a durometer and is arranged so that moving proximally along the length of the sheath body 102, the durometer increases. The coil reinforcement 130 can stretch out moving distally. The braid reinforcement 125 cinched directly over the coil reinforcement 130 can have variable pic count along its length so that the distal portion of the braid reinforcement 130 is less dense than a proximal portion of the braid reinforcement 130.

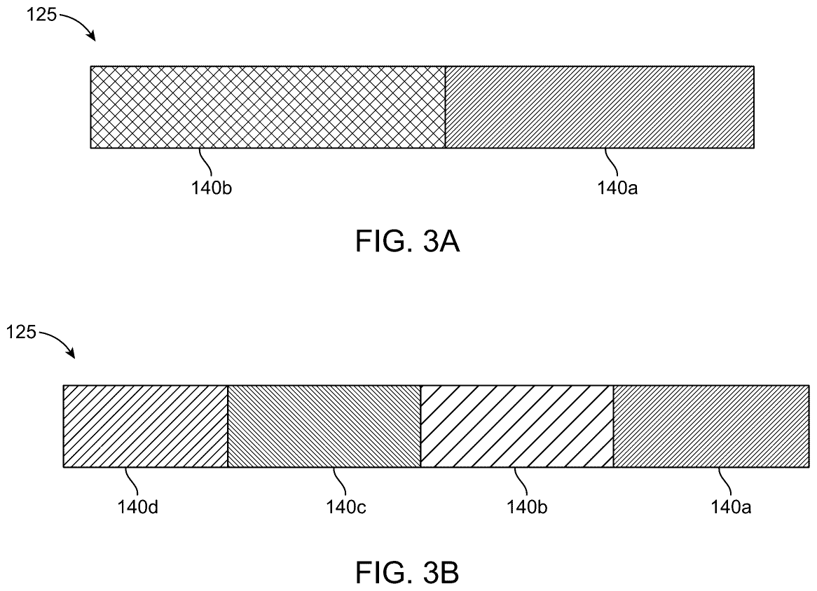

The braid reinforcement 125 can vary along its length in density thereby forming different braid zones 140 so that the braid positioned directly onto the coil has a variable pic count along its length. The braid reinforcement 125 can incorporate at least 2 braid zones 140, preferably greater than 2, including 3, 4, 5, or more braid zones 140 between the distal end and the proximal end of the sheath body 102. The braid zones 140 are formed in the single braid structure, but are formed to have differences in pics per inch (PPI). In an implementation, the braid reinforcement 125 includes a distal braid zone 140a connected to a proximal braid zone 140b (see FIG. 3A). The proximal braid zone 140b can have a lower PPI compared to the distal braid zone 140a such that the proximal braid zone 140b is generally looser than the tighter distal braid zone 140a. For example, the proximal braid zone 140b can have a PPI that is about 60 PPI to no greater than about 90 PPI and the distal braid zone 140a can have a PPI that is 90 PPI or greater, such as about 90-110 PPI. In another implementation, the braid reinforcement 125 includes four braid zones 140 including a first braid zone 140a along the distal end region of the sheath body 102 having a PPI of about 100-110 PPI, a second braid zone 140b moving proximally from the first braid zone 140a having a PPI of about 90-100, a third braid zone 140c moving proximally from the second braid zone 140b having a PPI of about 80-90 PPI, a fourth braid zone 140d along a proximal end region of the guiding sheath 100 having a PPI of about 60-70 PPI (see FIG. 3B). The first braid zone 140a can have a length of about 60-70 mm, the second braid zone 140b can have a length of about 90-100 mm, the third braid zone 140c can have a length of about 75-85 mm, and the fourth braid zone 140c can have a length of about 650-660 mm, depending on the working length of the sheath body 102.

The looser PPI regions (i.e., having lower PPI) of the proximal braid zone 140b can extend along a length that is greater than the tighter PPI regions (i.e., having higher PPI) of the distal braid zone 140a. For example, the proximal braid zone 140b can be about 70 cm or between about 60-80 cm and the distal braid zone 140a can be about 20 cm or between about 10-30 cm.

The first braid zone 140a can be even tighter and have higher PPI and the fourth braid zone 140d can be even looser and have lower PPI. The PPI can increase by about 30% between the fourth braid zone 140d and the next segment (i.e., third braid zone 140c). The PPI can increase by about 10% between the third braid zone 140c and the second braid zone 140b and can increase again by about 10% between the second braid zone 140b and the first braid zone 140a.

As illustrated in FIG. 1B, the distal edge of the final coil of the coil reinforcement 130 can be aligned substantially with the proximal edge of the distal radiopaque marker 111, preferably within about 2 mm or less. The radiopaque marker 111 is fully encapsulated between the inner liner layer 109 and the outer jacket layer 107. The radiopaque marker 111 preferably covers an end of the braid reinforcement 125 so that the end is fully encapsulated between the radiopaque marker 111 and the inner liner layer 109. The braid reinforcement 125 can cover a termination or final coil of the coil reinforcement 130. The final coil of the coil reinforcement 130 can be covered by a segment 127, such as a PET segment. The segment 127 can have a wall thickness of about 0.0005″-0.015″ and an inner diameter of about 0.112″-0.118″. The final coil of the coil reinforcement 130 is thus, fully encapsulated between the outer jacket layer 107, the braid reinforcement 125, and the PET segment 127 on an outer surface of the coil reinforcement 130 and the inner liner layer 109 on the inner surface of the coil reinforcement 130.

The sheath body 102 can include a hydrophilic coating along at least a portion of its length, along at least a portion of the distal end region of the sheath body 102. The coating of the external surface of the sheath body 102 with the material can reduce friction during use. The length of the coating can extend from the distal-most end of the sheath body 102 at least about 5 cm to about 10 cm proximal of the distal-most end.

The guiding sheath 100 illustrated in FIGS. 1A-1B can be used alone or as part of a sheath system. FIG. 2 illustrates optional components that can be used together with the guiding sheath 100. The sheath system 150 can incorporate one or more of a navigating catheter 300, an introducer dilator 400 for percutaneous entry into a vessel, and a hemostasis valve component 200.

The navigation catheter 300 and introducer dilator 400 can be designed to be compatible with an 0.035″ or similar sized guidewire. The navigation catheter 300 can have a Simmons shape or a Berenstein shape at the tip region or other similar shape designed to select a take-off from the aortic arch up into the carotid vessels. The guiding sheath 100 with the introducer dilator 400 is capable of passing through the skin and into a vessel to access the vessel lumen without the use of an introducer sheath and with minimum rollback at the distal tip. The guiding sheath 100 can also be inserted using an introducer sheath (not shown). The introducer can be 8F, and is preferably 9F (e.g., inner diameter of 0.123″ or 3.12 mm) to maintain the smallest access site penetration as possible while allowing for insertion of superbore catheters through the working lumen of the guiding sheath. An access site penetration or arteriotomy site that is kept small is important to allow the use of a vessel closure device that rapidly seals the artery puncture after the procedure. Smaller access sites reduce the time to hemostasis following the procedure. Despite this desire to decrease the access site penetration by minimizing the outer diameter, a large inner diameter is also desired so that large aspiration catheters and super-bore aspiration catheters can be advanced to the target treatment sites. A majority of clots are found in M1 cerebral vessels. An aspiration catheter that is closely matched to the diameter of the cerebral vessels with the clot is better at removing the clot in a single pass. The seemingly conflicting goals of a small outer diameter and a large inner diameter results in the guiding sheath having a thinner wall than conventional sheaths. The coil/braid reinforcement configurations described herein provide the thin-walled sheath having a large inner diameter with exceptional navigability, support, and kink resistance while minimizing the access site penetration.

The guiding sheath 100 can be packaged alone or with one or more optional components including the rotating hemostatic valve component 200, the navigating catheter 300, an introducer dilator 400, and/or an introducer sheath. Each can be provided with a standard luer lock fitting that is compatible with mating devices.

### Methods of Use

A method of accessing and treating a vessel is now described. The sheath systems described herein can be used for the introduction of interventional devices into the peripheral vasculature and, preferably, the neurovasculature. The sheath system can include the long guiding sheath 100, an optional select catheter (e.g., Berenstein tip select catheter), and an optional navigation catheter. The sheath system can optionally incorporate a hemostasis valve component 200. The inner lumens of each of the sheath system components can be flushed, for example, with heparinized saline to hydrate the catheters before use.

The optional introducer dilator 400 can be fully inserted into the guiding sheath 100 for insertion at an access site directly into the vessel or using an introducer sheath previously positioned within the vessel. The introducer dilator 400 can include a luer that is designed to press fit into the corresponding component of the guiding sheath 100. The guiding sheath 100 can be inserted initially into the vessel using the introducer dilator 400 for percutaneous entry into the vessel over a guidewire. The guiding sheath 100 and dilator assembly can be advanced over the guidewire in the vessel to the intended position prior to removing the dilator from the guiding sheath 100. In the case where an introducer sheath is used, the appropriately sized introducer sheath (preferably 9 Fr) can be placed in the vessel using standard techniques and the guiding sheath 100 and dilator assembly inserted through the introducer sheath and advanced to the intended position prior to removing the dilator from the guiding sheath 100. The introducer dilator 400 can be exchanged for a navigation catheter 300 and advanced towards a target location.

The guiding sheath 100 can be inserted at an access site of a vessel (e.g., femoral artery, radial artery, or other access site) and advanced to a target site, such as a region of the internal carotid artery, such as the carotid bulb. The navigation catheter 300 can lead the guiding sheath 100 to the target site and once placement is achieved, the guiding sheath 100 can be advanced over the navigation catheter 300 and parked into place within the carotid bulb. Other access sites and target parking locations are considered as well. For example, if the access site of a vessel is a vein, the guiding sheath 100 can be inserted into a femoral vein in the groin of the patient, and advanced at least to a level of the internal jugular vein. The guiding sheath 100 can also be positioned to sites distal to the carotid bulb or internal jugular vein depending on the location of the treatment site. For example, in the case of treating the superior sagittal sinus, advancing the distal end of the guiding sheath 100 to a region of the transverse sinus may improve outcomes.

The guiding sheath 100 can be placed on continuous flush with the hemostasis valve component 200. The guiding sheath 100 having an attached hemostasis valve component 200 can be manually flushed, such as with heparinized saline, to flush the line to the guiding sheath 100. The navigation catheter 300 can be inserted through the guiding sheath 100 to assist in placement of the guiding sheath 100 at an intended location for support of neurovascular catheters, such as in the proximal ICA or distal carotid, under fluoroscopic guidance using standard endovascular techniques. Optionally, the distal end of the guide sheath can be advanced to the distal ICA or proximal M1. Low-magnification imaging for viewing can be used to monitor distal tip advancement and proximal segment stability of the guiding sheath 100. Distal “pushback” and proximal prolapse into the aortic arch can be monitored so that timely adjustments are made to return the guiding sheath to the desired location.

The navigation catheter 300 can have an outer diameter sized to match an inner diameter of the guiding sheath 100, particularly, the inner diameter at the distal end region to minimize the ledge effect at the distal face of the guiding sheath 100. The guiding sheath 100 can be between 6F to 10F, preferably an 8F size, and is at least 80 cm or 90 cm long. The optional navigation catheter for the 8F guiding sheath 100 can have an outer diameter up to about 0.104″ to provide sheath support and reduce ledge effect.

The stiffness profile of the navigation catheter 300 can also provide the softer, more flexible distal end region of the guiding sheath 100 and its soft distal tip 106 with sufficient support to deliver to target sites. Fluoroscopy can be used to watch for any distal “pushback” and proximal prolapse of the distal end region of the guiding sheath 100 back into the aortic arch in case adjustments are desired. Upon positioning the distal end of the guiding sheath 100 at the target location, the navigation catheter 300 can be removed from the lumen of the guiding sheath 100. Another device can be inserted through the hemostasis valve component 200 of the guiding sheath 100 and advanced through the working lumen of the guiding sheath 100 for treatment of a vessel. The device inserted through the guiding sheath 100 can vary including any of a variety of delivery catheter for delivery and deployment of a stent, flow diverter, or other expandable device, or for delivery of a fluid including a fluid treatment catheter or an aspiration catheter.

### Materials

One or more components of the catheters and catheter systems described herein may include or be made from a variety of materials including one or more of a metal, metal alloy, polymer, a metal-polymer composite, ceramics, hydrophilic polymers, polyacrylamide, polyethers, polyamides, polyethylenes, polyurethanes, copolymers thereof, polyvinyl chloride (PVC), PEO, PEO-impregnated polyurethanes, such as Hydrothane, Tecophilic polyurethane, Tecothane, PEO soft segmented polyurethane blended with Tecoflex, thermoplastic starch, PVP, and combinations thereof, and the like, or other suitable materials.

Some examples of suitable cut-tube or flat metal material includes Nitinol, Layered tube with Nitinol on outside and inner core of radiopaque material, such as tantalum, platinum, iridium, gold, alloy etc. Additionally, material could be cobalt, cobalt alloy, or stainless steel.

Some examples of suitable metals and metal alloys include stainless steel, such as 304V, 304L, and 316LV stainless steel; mild steel; nickel-titanium alloy, such as linear-elastic and/or super-elastic Nitinol; other nickel alloys, such as nickel-chromium-molybdenum alloys (e.g., UNS: N06625, such as INCONEL® 625, UNS: N06022, such as HASTELLOY® C-22®, UNS: N10276, such as HASTELLOY® C276®, other HASTELLOY® alloys, and the like), nickel-copper alloys (e.g., UNS: N04100, such as MONEL® 100, NICKELVAC® 100, NICORROS® 100, and the like), nickel-cobalt-chromium-molybdenum alloys (e.g., UNS: R30035, such as MP35-N® and the like), nickel-molybdenum alloys (e.g., UNS: N10665, such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003, such as ELGILOY®, PHYNOX®, and the like); platinum enriched stainless steel; titanium; combinations thereof; and the like; or any other suitable material and as described elsewhere herein.

Inner liner materials of the catheters described herein can include low friction polymers, such as PTFE (polytetrafluoroethylene) or FEP (fluorinated ethylene propylene), PTFE with polyurethane layer (Tecoflex). Reinforcement layer materials of the catheters described herein can be incorporated to provide mechanical integrity for applying torque and/or to prevent flattening or kinking, such as metals including stainless steel, Nitinol, Nitinol braid, helical ribbon, helical wire, cut stainless steel, or the like, or stiff polymers, such as PEEK. Reinforcement fiber materials of the catheters described herein can include various high tenacity polymers like Kevlar, polyester, meta-para-aramide, PEEK, single fiber, multi-fiber bundles, high tensile strength polymers, metals, or alloys, and the like. Outer jacket materials of the catheters described herein can provide mechanical integrity and can be contracted of a variety of materials, such as polyethylene, polyurethane, PEBAX, nylon, Tecothane, and the like. Other coating materials of the catheters described herein include paralene, Teflon, silicone, polyimide-polytetrafluoroetheylene, and the like. The inner liner may further include different surface finishes, such as dimples, bumps, ridges, troughs. The surface finishes may be randomly disposed, linearly disposed, spirally disposed, or otherwise disposed using a specific pattern along the length of the catheter. It is further contemplated that the inner liner may include a mixture of different surface finishes, for example, one section may have dimples, another section may have troughs, etc. Additionally, the surface finish may be incorporated along the entire length of the catheter or only in sections of the catheter. It is also contemplated that the inner liner may further include an electrosprayed layer, whereby materials could be incorporated into the inner liner. Examples of materials can include low friction materials as described above. Alternatively, the electrosprayed or electrospun layer may incorporate a beneficial agent that becomes free from the coating when exposed to blood, or to compression from a clot, for example, the beneficial agent may be a tissue plasminogen activator (tPA), or heparin encased in alginate.

Implementations describe catheters and catheter systems and methods to deliver catheters to target anatomies. However, while some implementations are described with specific regard to delivering catheters to a target vessel of a neurovascular anatomy, such as a cerebral vessel, the implementations are not so limited and certain implementations may also be applicable to other uses. For example, the catheters can be adapted for delivery to different neuroanatomies, such as subclavian, vertebral, carotid vessels as well as to the coronary anatomy or peripheral vascular anatomy, to name only a few possible applications. It should also be appreciated that although the systems described herein are described as being useful for treating a particular condition or pathology, that the condition or pathology being treated may vary and are not intended to be limiting.

### EXAMPLES

The deliverability, support, and kink resistance characteristics of guiding sheaths were measured. The guiding sheaths tested included a conventional guiding sheath having a coil-over-braid reinforcement arrangement and fewer material transitions from distal tip to proximal end region (referred to in Examples 1-3 as “Sheath 1.5”) compared to a guiding sheath having a coil-under-braid reinforcement arrangement as shown in FIGS. 1A-1B and more material transitions from distal tip to proximal end region (referred to in Examples 1-3 as “Sheath 2.0”).

The braid of Sheath 1.5 was a stainless steel rectangular wire and the coil was a Nitinol ribbon. The braid of Sheath 2.0 was a stainless steel round wire and the coil was a stainless steel ribbon. Both Sheath 1.5 and Sheath 2.0 had the same inner diameter, outer diameter, and working lengths. Both Sheath 1.5 and Sheath 2.0 were coated with the same lubricious coating.

### Example 1

The flexibility characteristics of the guiding sheaths were measured using a 3-point Bending Test method. Each test sheath was marked to identify test segments to be tested along a length of the sheath.

Sheath 1.5 was tested along 5 different test segments including a first segment that was 0-4 cm from the distal-most end of the catheter, a second segment that was 4-6 cm from the distal-most end, a third segment that was 6-8 cm from the distal-most end, a fourth segment that was 8-13 cm from the distal-most end, and a fifth segment that was at least 13 cm from the distal-most end and greater.

Sheath 2.0 was tested along 7 different test segments including a first segment that was 0-2.5 cm from the distal-most end of the catheter, a second segment that was 2.5-5 cm from the distal-most end, a third segment that was 5-7.5 cm from the distal-most end, a fourth segment that was 7.5-10 cm from the distal-most end, a fifth segment that was 10-22 cm from the distal-most end, a sixth segment that was 22-26 cm from the distal-most end, and a seventh segment that was at least 26 cm from the distal-most end and greater. Sheath 2.0 was tested along a greater number of segments compared to Sheath 1.5 due to those test sheaths having a greater number of material transitions along the length of the sheath. The material transitions included transitions between outer jacket layer segments formed of materials having a hardness different from a material having a material hardness of an adjacent outer jacket layer segment.

The test sheaths were pre-conditioned and tested at 37° C. A calibrated 10 N load cell was attached to the upper crosshead of an Instron Universal Testing Machine and a 500 N load cell attached and secured by pneumatic grip. Test sheaths were supported within protective tubing held within separate vice grips for support. The test sheaths were loaded at substantially the same height through the protective tubing so a desired test section was centered under an anvil of the Testing Machine. The vice grips were positioned about 2 cm apart and the crosshead position adjusted downwards towards the test section until the anvil of the crosshead was arranged just above and out of contact with the test sheath. The crosshead position was adjusted downwards until a small, consistent positive load was measured and the test sheath was in full contact with the bottom of the protecting tubing. The gauge length was 3.00 mm, Deflection distance was 2.50 mm, Force was measured at 2.00 mm, and Crosshead speed was 25.00 mm/min.

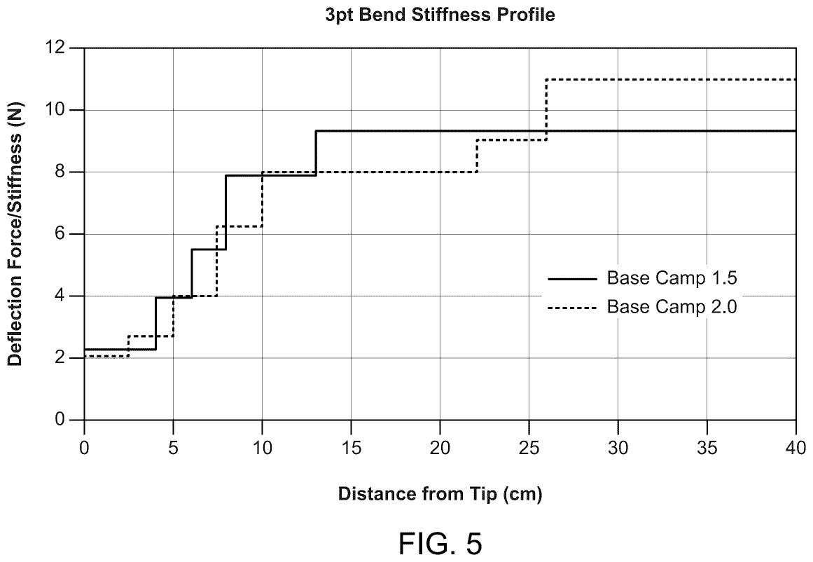

The stiffness results for each unit were averaged together to define a stiffness value for each test segment. The test segment stiffnesses were combined to create the stiffness profile of the full test sheath. Table 1 below illustrates the average force in Newtons (N) along segments of the sheaths tested and FIG. 5 illustrates the stiffness profile.

TABLE 1

| | | | |
|---|---|---|---|
| Segments | | Segments | |
| (distance | Average Force | (distance | Average Force |
| from distal- | (Newtons) | from distal- | (Newtons) |
| most end) | Sheath 1.5 | most end) | Sheath 2.0 |

| | | | | | |
|---|---|---|---|---|---|
| 0-4 | cm | 2.29 | 0-2.5 | cm | 2.09 |
| 4-6 | cm | 3.99 | 2.5-5 | cm | 2.71 |
| 6-8 | cm | 5.46 | 5-7.5 | cm | 4.04 |
| 8-13 | cm | 7.89 | 7.5-10 | cm | 6.27 |
| 13+ | cm | 9.34 | 10-22 | cm | 7.99 |
| | | | 22-26 | cm | 9.08 |
| | | | 26+ | cm | 11.01 |

Sheath 2.0 with the coil under the braid and a greater number of material transitions along its length had a more flexible distal end region and a stiffer proximal end region compared to a conventional Sheath 1.5 having a braid under the coil and fewer material transitions. Sheath 2.0 also had a more gradual transition between the distal and proximal ends provided by these material transitions indicating better support and push response compared to Sheath 1.5. Sheath 2.0 was about 26% more flexible along the distal segment compared to Sheath 1.5 and was about 18% more supportive (stiffer) along the proximal segment compared to Sheath 1.5. The average force in Newtons of Sheath 2.0 within the 10-15 cm portion was 4× the average force in Newtons at the distal end and the Sheath 2.0 incorporated 5 transitions in jacket materials. The average force in Newtons of Sheath 2.0 at the proximal end was 27% greater than the average force in Newtons of the sheath at the 10-15 cm portion. The total difference between the distal flexibility and the proximal flexibility was greater than 5×.

Trackability of the guiding sheaths were measured using a Sheath Track Test. An anatomical silicone model was used to simulate access of middle cerebral arteries from a femoral artery access site. To prepare the model, a 9 Fr introducer sheath with side tube was attached to a barb at the right femoral artery of the model using a piece of tubing. An introducer sheath with side tube was attached to a barb at the external carotid artery of the head/neck model using a second piece of tubing. All unused barbs and vessels of the model were occluded. The model was positioned on an E-0310 MSI Track Tester such that the introducer sheath lines up with the track tester roller assembly. The model was set up with the ends of the inflow and outflow tubing placed into a water bath containing a mild detergent. A portion of the inflow tubing was placed into a peristaltic pump. All air was purged from the model before and after testing. The descending aorta of the model was clamped with sufficient clearance so that the test sheaths follow the carotid takeoff.

### Example 2

Track testing was conducted on 3 each of the Sheath 1.5 and Sheath 2.0. The Advantage Glidewire (Terumo) was used to facilitate delivery of the navigation catheter size-matched to the sheath. The test sheaths as well as the guidewire and navigation catheter used in testing were prepared by hydrating and flushing. The guidewire was inserted into the flushed navigation catheter, and the navigation catheter was inserted into the flushed test sheath and the hubs were fixed together. The distal tip of the guidewire was advanced past the carotid bifurcation of the model. The tip of the navigation catheter was advanced to the carotid takeoff and the tip position marked on the model for consistency as needed. The proximal end of the guidewire was fixed using toggle clamps on a holding tray. The peristaltic pump was powered off before track testing of the test sheath began. The test sheath and navigation catheter were tracked together using the track tester. The sheath and navigation catheter were advanced distally through the model 20 cm as a first step and paused for about 10-15 seconds, which allowed time to disconnect the test sheath from the navigation catheter. The test sheath was advanced distally over the navigation catheter 8 cm into the common carotid artery of the model as a second step before pausing again for about 5 seconds. The clamps holding the navigation catheter hub and the proximal end of the guidewire were disengaged and then the track testing withdrew the components the distance advanced (i.e., 28 cm) as a third step. Each step was performed at a speed of 50 cm/min prepared at encoder pressure of 30 psi and motor pressure of 80 psi.

Table 2 below illustrates the average Maximum Delivery Force in grams (g) and the average Maximum Withdrawal Force in grams (g) of the sheaths tested.

TABLE 2

| | Maximum Delivery | Maximum Withdrawal |
|---|---|---|
| | Force (g) | Force (g) |
| Sheath 1.5 | 895.2 | 722.3 |
| Sheath 2.0 | 613.1 | 594.7 |

Sheath 2.0 had a lower average delivery and withdrawal force compared to Sheath 1.5 indicating better trackability provided by the greater material transitions, more flexible distal tip, greater proximal stiffness, and the coil under the braid construction. The delivery force needed for sheath advancement in Sheath 2.0 was about 32% less than the delivery force needed for advancement of Sheath 1.5 and the withdrawal force needed for removal of Sheath 2.0 was about 18% less compared to the withdrawal force needed to remove Sheath 1.5. Sheath 2.0 included a tight-pitch stainless steel coil and dual-layer strain relief providing the sheath with both distal and proximal support during advancement. Sheath 2.0 included seven different copolymer zones creating a smoother overall transition profile and a softer distal section compared to Sheath 1.5 that provided enhanced maneuverability, even in complex anatomy.

### Example 3

The kink resistance characteristics of the guiding sheaths were measured using a vise jaw gap test to assess the minimum gap distance before kinking occurred. Sheath 1.5 and Sheath 2.0 were each tested along a test segment that was about 10-15 cm from the distal end of the catheter, which is typically the portion of the catheter that traverses the most tortuous anatomy of intracranial vessels and at risk for kinking. Each test segment was curved into a loop so that an apex of the loop was positioned squarely between jaws of a vise. The jaws of the vise were slowly moved towards one another reducing the gap between the jaws creating a tighter curve to the loop. The apex of the loop was observed for evidence of kinking, such as a sharp point forming within some portion of the apex (e.g., a radially inward part of the curve), and/or collapse of the lumen. Once the test segment kinked, the motion of the vise was stopped and the distance between the jaws was measured with calipers. Five samples of each of Sheath 1.5 and Sheath 2.0 were tested and jaw gap distances averaged (see Table 3 below). Sheath 2.0 had a higher kink resistance (about 41% higher) compared to Sheath 1.5. Kinking did not occur until the gap between the jaws was as small as about 26.8 mm apart and the test loop between the jaws forced into a tighter curve shape compared to Sheath 1.5, which kinked when the gap between the jaws was just 45.5 mm and the test loop between the jaws was urged into a curve shape that was not as tight (e.g., had a larger radius of curvature). Sheath 2.0 with the braid reinforcement positioned over the coil reinforcement was able to achieve a tighter curved shape without kinking compared to Sheath 1.5 having a coil reinforcement over the braid reinforcement.

The kink resistance of the guiding sheath 100 within the distal end region (e.g., distal-most 10-15 cm) of the sheath 100 with the braid reinforcement positioned over the coil reinforcement is greater than the kink resistance of a guiding sheath with the coil reinforcement over the braid reinforcement. The kink resistance as measured by vise jaw gap distance is no greater than 45 mm, no greater than 40 mm, and preferably no greater than 30 mm.

TABLE 3

| | Jaw gap distance (mm) |
|---|---|
| Sheath 1.5 | 45.5 |
| Sheath 2.0 | 26.8 |

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference, and are not intended to limit the use or orientation of the catheters and/or delivery systems to a specific configuration described in the various implementations.

The word “about,” “approximately,” and “substantially,” mean a range of values including the specified value, which a person of ordinary skill in the art would consider reasonably similar to the specified value. In embodiments, about means within a standard deviation using measurements generally acceptable in the art. In embodiments, “about” means a range extending to +/−10% of the specified value. In embodiments, about includes the specified value. One inch or 1″ corresponds to 2.54 cm (SI-units).

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The components of the systems disclosed herein may be packaged together in a single package or separately. The finished package would be sterilized using sterilization methods such as Ethylene oxide or radiation and labeled and boxed. Instructions for use may also be provided in-box or through an internet link printed on the label.

What is claimed is:

1. An intravascular guiding sheath for facilitation of intraluminal medical procedures within a neurovasculature, the guiding sheath comprising:
a variable stiffness sheath body having a single working lumen extending through the sheath body from a proximal end region to a distal opening at a distal end region of the sheath body, the sheath body having a length between a proximal opening and the distal opening, wherein the working lumen has a minimum inner diameter that is at least 0.080″; and
a proximal hub luer at the proximal end region of the sheath body,
wherein the sheath body comprises a wall having a wall thickness from the proximal end region to the distal end region, wherein the wall comprises an outer jacket layer, an inner liner layer, and a reinforcement layer between the outer jacket layer and the inner liner layer,
wherein the reinforcement layer comprises a coil reinforcement wound over the inner liner layer and a braid reinforcement positioned directly onto the coil reinforcement that has a variable pic count, and
wherein the outer jacket layer has a plurality of outer jacket segments along the length of the sheath body, each outer jacket segment of the plurality of outer jacket segments having a durometer and arranged so that moving proximally along the length of the sheath body the durometer of the plurality of outer jacket segments increases.

2. The intravascular guiding sheath of claim 1, wherein the plurality of outer jacket segments is at least six outer jacket segments along the length of the sheath body.

3. The intravascular guiding sheath of claim 2, wherein the at least six outer jacket segments comprises a distal set of outer jacket segments and a proximal set of outer jacket segments.

4. The intravascular guiding sheath of claim 3, wherein the distal set of outer jacket segments comprises four outer jacket segments, each of the four outer jacket segments has a length, wherein the length of each of the four outer jacket segments are substantially the same length to each other.

5. The intravascular guiding sheath of claim 4, wherein the proximal set of outer jacket segments comprises at least two outer jacket segments, each of the at least two outer jacket segments has a length, wherein the length of each of the at least two outer jacket segments is longer than the length of each of the four outer jacket segments.

6. The intravascular guiding sheath of claim 1, wherein the inner liner layer and the outer jacket layer are polymeric and the reinforcement layer is metal.

7. The intravascular guiding sheath of claim 1, wherein the coil reinforcement is stainless steel.

8. The intravascular guiding sheath of claim 1, wherein the braid reinforcement comprises at least four zones of different density including a first braid zone along the distal end region of the sheath body, a second braid zone located proximal to the first braid zone, a third braid zone located proximal to the second braid zone, and a fourth braid zone located proximal to the third braid zone and extending along the proximal end region of the sheath body.

9. The intravascular guiding sheath of claim 8, wherein each of the first braid zone, second braid zone, third braid zone, and fourth braid zone has a pics per inch (PPI), wherein the PPI increases by about 30% between the fourth braid zone and the third braid zone, wherein the PPI increases by about 10% between the third braid zone and the second braid zone, and the PPI increases by about 10% between the second braid zone and the first braid zone.

10. The intravascular guiding sheath of claim 1, wherein the wall thickness is substantially uniform.

11. The intravascular guiding sheath of claim 1, wherein the wall thickness is about 0.006″ to about 0.012″.

12. The intravascular guiding sheath of claim 1, further comprising a double layer strain relief along a junction between the proximal end region of the sheath body and the proximal hub luer.

13. The intravascular guiding sheath of claim 12, wherein the double layer strain relief comprises an outer layer formed of a first polymer material and an inner layer formed of a second polymer material.

14. An intravascular guiding sheath for facilitation of intraluminal medical procedures within a neurovasculature, the guiding sheath comprising:
a variable stiffness sheath body having a single working lumen extending through the sheath body from a proximal end region to a distal opening at a distal end region of the sheath body, the sheath body having a length between a proximal opening and the distal opening, wherein the working lumen has a minimum inner diameter that is at least 0.080″; and
a proximal hub luer at the proximal end region of the sheath body for connection to a fluid delivery or aspiration source,
wherein the sheath body comprises a wall having a wall thickness, wherein the wall comprises an outer jacket layer, an inner liner layer, and a reinforcement layer between the outer jacket layer and the inner liner layer,
wherein the reinforcement layer comprises a stainless-steel coil wound over the inner liner layer and a braid positioned directly onto the coil that has a variable pic count,
wherein the outer jacket layer has a plurality of outer jacket segments along the length of the sheath body, each outer jacket segment of the plurality of outer jacket segments having a durometer and arranged so that moving proximally along the length of the sheath body the durometer of the plurality of outer jacket segments increases, and
wherein the braid comprises at least three braid zones having different pics per inch (PPI) from one another, wherein a distal-most braid zone of the at least three braid zones is overlaid by at least three outer jacket segments of the plurality of outer jacket segments of the outer jacket layer.

15. The intravascular guiding sheath of claim 14, wherein the plurality of outer jacket segments is at least six outer jacket segments along the length of the sheath body.

16. The intravascular guiding sheath of claim 15, wherein the at least six outer jacket segments comprises a distal set of outer jacket segments and a proximal set of outer jacket segments.

17. The intravascular guiding sheath of claim 16, wherein the distal set of outer jacket segments comprises four outer jacket segments, each of the four outer jacket segments has a length, wherein the length of each of the four outer jacket segments are substantially the same length to each other.

18. The intravascular guiding sheath of claim 17, wherein the proximal set of outer jacket segments comprises at least two outer jacket segments, each of the at least two outer jacket segments has a length, wherein the length of each of the at least two outer jacket segments is longer than the length of each of the four outer jacket segments.

19. The intravascular guiding sheath of claim 14, wherein the wall thickness is substantially uniform.

20. The intravascular guiding sheath of claim 14, wherein the wall thickness is about 0.006″ to about 0.012″.

21. The intravascular guiding sheath of claim 14, further comprising a double layer strain relief along a junction between the proximal end region of the sheath body and the proximal hub luer.

22. The intravascular guiding sheath of claim 21, wherein the double layer strain relief comprises an outer layer formed of a first polymer material and an inner layer formed of a second polymer material.

23. A system comprising an intravascular guiding sheath of claim 14 and a navigation catheter.

24. A method of using the intravascular guiding sheath of claim 14.

25. An intravascular guiding sheath for facilitation of intraluminal medical procedures within a neurovasculature, the guiding sheath comprising:
a variable stiffness sheath body having a single working lumen extending through the sheath body from a proximal end region to a distal opening at a distal end region of the sheath body, the sheath body having a length between a proximal opening and the distal opening, wherein the working lumen has a minimum inner diameter; and
a proximal hub luer at the proximal end region of the sheath body,
wherein the sheath body comprises a wall having a wall thickness from the proximal end region to the distal end region, wherein the wall comprises an outer jacket layer, an inner liner layer, and a reinforcement layer between the outer jacket layer and the inner liner layer,
wherein the reinforcement layer comprises a coil reinforcement wound over the inner liner layer and a braid reinforcement positioned directly onto the coil reinforcement that has a variable pic count, and
wherein the outer jacket layer has at least six outer jacket segments along the length of the sheath body, each outer jacket segment of the at least six outer jacket segments having a durometer and arranged so that moving proximally along the length of the sheath body the durometer of the outer jacket segments increases.

26. The guiding sheath of claim 25, wherein the variable stiffness sheath body is 6 French up to 10 French.

27. The guiding sheath of claim 25, wherein the braid reinforcement compresses the coil reinforcement against the inner liner layer.

28. The guiding sheath of claim 25, wherein a distal portion of the sheath body has a kink resistance as measured by vise jaw gap distance is no greater than 40 mm, no greater than 35 mm, or no greater than 30 mm.
